(19) United States
(12) Patent Application Publication
(10) Pub. No.: US 2026/0087594 A1
(43) Pub. Date: Mar. 26, 2026

(54) METHOD AND SYSTEM FOR STITCHING ANATOMICAL IMAGES

(71) Applicant: L&T TECHNOLOGY SERVICES LIMITED, Chennai (IN)

(72) Inventors: AKSHAYA BABU, Kochi (IN); ANUSREE KAREEPARAMBATH, Kozhikode (IN)

(21) Appl. No.: 19/311,036

(22) Filed: Aug. 27, 2025

(30) Foreign Application Priority Data
Sep. 20, 2024 (IN) 202441071153

(51) Int. Cl. G06T 5/50; G06T 5/40; G06T 5/90; G06V 10/24; G06V 10/25; G06V 10/60; G16H 30/40

(52) U.S. Cl. CPC G06T 5/50; G06T 5/40; G06T 5/90; G06V 10/24; G06V 10/25; G06V 10/60; G16H 30/40; G06T 2207/20221

(57) ABSTRACT

This disclosure relates to method and system for stitching anatomical images. The method includes receiving a first image and a second image. For each of a plurality of pixel rows of the first image selected from bottom to top, and for each of a plurality of pixel rows of the second image selected from top to bottom, the method includes cumulatively creating a region of interest (ROI) from a first image pixel row with a second image pixel row until the ROI includes each of the plurality of pixel rows of each of the first image and the second image, calculating an intensity difference between a plurality of first image ROI pixels and a corresponding plurality of second image ROI pixels, and determining a mean intensity difference for the ROI based on the intensity difference to identify an overlapping ROI.

### TECHNICAL FIELD

This disclosure relates generally to medical imaging, and more particularly to method and system for stitching anatomical images.

### BACKGROUND

Medical imaging techniques (for example, Magnetic Resonance Imaging (MRI), X-Ray, Computed Tomography (CT), etc.) enable capturing of detailed images of internal structures (such as organs, bones, muscles, and blood vessels) of an anatomical region of a body. For diagnosing and treating one or more conditions affecting an anatomical region (for example, the spine), the anatomical image showing a comprehensive view of the anatomical region may be required. Such an anatomical region may be constituted by a plurality of component anatomical regions. In case of the spine, understanding the continuity and relationships between different vertebral segments may be vital. The plurality of component anatomical regions of the spine may include a cervical region, a thoracic region, and a lumbar region. However, the images of the plurality of component anatomical regions may be acquired separately due to limitations in a field of view of a medical imaging device.

A comprehensive image of the anatomical image may facilitate comprehensive visualization of multiple anatomical regions, improved diagnostic precision, enhanced treatment planning, and improved monitoring and follow-up. To this end, many image stitching techniques have been developed that stitch (or join) the images of the plurality of component anatomical regions to obtain the comprehensive image. Conventional image stitching techniques, however, require manual intervention and face challenges in seamlessly stitching the images together. For example, there may be a duplication of redundant overlapping regions at joining ends of two images. This may lower accuracy and reliability of the comprehensive image. To overcome the problem of the duplication of the overlapping regions, conventional image stitching techniques require a marker to identify a location of points of reference for comparing overlapping regions of the images. The marker in two images are manually observed, adding to the complexity of the image stitching process.

Thus, the present invention is directed to overcome one or more limitations stated above or any other limitations associated with the known arts.

### SUMMARY

In one embodiment, a method for stitching anatomical images is disclosed. In one example, the method may include receiving a first image and a second image. The first image may correspond to a first anatomical region and the second image may correspond to a second anatomical region. The first image may include an overlapping region with the second image. For each of a plurality of pixel rows of the first image selected from bottom to top, and for each of a plurality of pixel rows of the second image selected from top to bottom, the method may further include cumulatively creating a region of interest (ROI) from a first image pixel row with a second image pixel row until the ROI may include each of the plurality of pixel rows of each of the first image and the second image. For each of a plurality of pixel rows of the first image selected from bottom to top, and for each of a plurality of pixel rows of the second image selected from top to bottom, the method may further include calculating an intensity difference between each of a plurality of first image ROI pixels and each of a corresponding plurality of second image ROI pixels. For each of a plurality of pixel rows of the first image selected from bottom to top, and for each of a plurality of pixel rows of the second image selected from top to bottom, the method may further include determining a mean intensity difference for the ROI based on the calculated intensity difference. The method may further include identifying an overlapping ROI from a plurality of ROIs based on the mean intensity difference. The overlapping ROI may include a minimum mean intensity difference.

In one embodiment, a system for stitching anatomical images is disclosed. In one example, the system may include a processor and a computer-readable medium communicatively coupled to the processor. The computer-readable medium may store processor-executable instructions, which, on execution, may cause the processor to receive a first image and a second image. The first image may correspond to a first anatomical region and the second image may correspond to a second anatomical region. The first image may include an overlapping region with the second image. For each of a plurality of pixel rows of the first image selected from bottom to top, and for each of a plurality of pixel rows of the second image selected from top to bottom, the processor-executable instructions, on execution, may further cause the processor to cumulatively create an ROI from a first image pixel row with a second image pixel row until the ROI may include each of the plurality of pixel rows of each of the first image and the second image. For each of a plurality of pixel rows of the first image selected from bottom to top, and for each of a plurality of pixel rows of the second image selected from top to bottom, the processor-executable instructions, on execution, may further cause the processor to calculate an intensity difference between each of a plurality of first image ROI pixels and each of a corresponding plurality of second image ROI pixels. For each of a plurality of pixel rows of the first image selected from bottom to top, and for each of a plurality of pixel rows of the second image selected from top to bottom, the processor-executable instructions, on execution, may further cause the processor to determine a mean intensity difference for the ROI based on the calculated intensity difference. The processor-executable instructions, on execution, may further cause the processor to identify an overlapping ROI from a plurality of ROIs based on the mean intensity difference. The overlapping ROI may include a minimum mean intensity difference.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of an exemplary system for stitching anatomical images, in accordance with some embodiments.

FIG. 2 illustrates a functional block diagram of a memory of a computing device configured to stitch anatomical images, in accordance with some embodiments.

FIG. 3 illustrates a flow chart of an exemplary process for pre-processing anatomical images, in accordance with some embodiments.

FIG. 4 illustrates a flow chart of a detailed exemplary process for pre-processing anatomical images, in accordance with an embodiment.

FIG. 5 illustrates a flow chart of an exemplary process for identifying an overlapping region of interest (ROI) between anatomical images, in accordance with some embodiments.

FIGS. 6A-C are schematic diagrams of cumulative creation of ROIs from anatomical images, in accordance with some embodiments.

FIG. 7 illustrates a flow chart of a detailed exemplary process for identifying an overlapping ROI between anatomical images, in accordance with some embodiments.

FIG. 8 illustrates a flow chart of an exemplary process for stitching anatomical images, in accordance with some embodiments.

FIGS. 9A-C are schematic diagrams of generation of difference images from shifted alignments of anatomical images, in accordance with some embodiments.

FIGS. 10A and 10B illustrate a flow chart of a detailed exemplary process for identifying horizontal shift between anatomical images, in accordance with an embodiment.

FIGS. 11A-C are schematic diagrams of blending of anatomical images, in accordance with some embodiments.

FIG. 12 illustrates a flow chart of a detailed exemplary process for blending anatomical images, in accordance with some embodiments.

FIG. 13 is a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

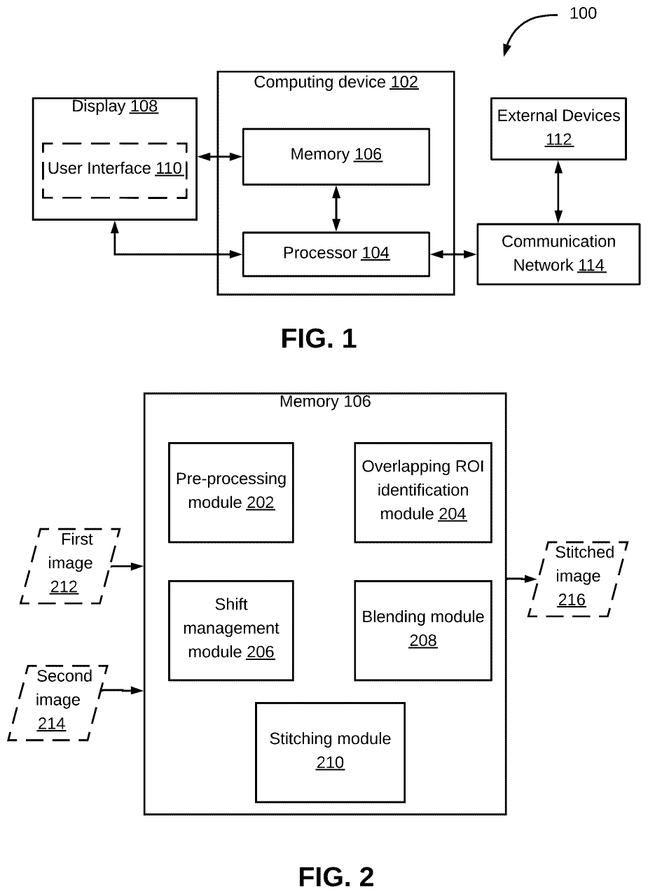

Referring now to FIG. 1, an exemplary system 100 for stitching anatomical images is illustrated, in accordance with some embodiments. A plurality of anatomical images (for example, Magnetic Resonance Imaging (MRI) scan images, Computed Tomography (CT) scan images, X-Ray images, or the like) may be captured through a medical imaging device (for example, an MRI scanner, a CT scanner, an X-ray machine, or the like). Each of the plurality of anatomical images may enable visualization of an anatomical region of a body of a patient. Examples of the anatomical region may include, but are not limited to, a cervical region, a thoracic region, and a lumbar region.

Stitching (i.e., an image stitching technique), as defined herein, may refer to joining two or more anatomical images corresponding to two or more anatomical regions. It should be noted that the two or more anatomical regions may be in continuity in the body. By way of an example, the two or more anatomical regions may be the cervical region, the thoracic region, and the lumbar region. As will be appreciated, the cervical region, the thoracic region, and the lumbar region are components of a spine region (a larger anatomical region). Thus, upon stitching images corresponding to the cervical region, the thoracic region, and the lumbar region, a stitched image may be obtained corresponding to the spine region. The stitched image may facilitate understanding of a continuity and a relationship between different components of the larger anatomical region. In case of the spine region, the components correspond to different vertebral segments of the spine.

The system 100 may include a computing device 102 (for example, a server, a desktop, a laptop, a notebook, a netbook, a tablet, a smartphone, a mobile phone, or any other computing device), in accordance with some embodiments of the present disclosure. The computing device 102 may stitch the anatomical images received as an input. The computing device 102 may process the anatomical images to identify an overlapping region between each pair of the anatomical images. The computing device 102 may further seamlessly stitch each pair of the anatomical images based on the identified overlapping region.

As will be described in greater detail in conjunction with FIGS. 2-12, the computing device 102 may receive a first image and a second image. The first image may correspond to a first anatomical region and the second image may correspond to a second anatomical region. The first image may include an overlapping region with the second image. For each of a plurality of pixel rows of the first image selected from bottom to top, and for each of a plurality of pixel rows of the second image selected from top to bottom, the computing device 102 may further cumulatively create a region of interest (ROI) from a first image pixel row with a second image pixel row until the ROI may include each of the plurality of pixel rows of each of the first image and the second image. A pixel row may be a row of a predefined pixel height (for example, 1 pixel, 2 pixels, etc.), extending horizontally from one edge of an image to an opposite edge. Thus, two pixel rows (one from each of the first image and the second image may be added to the ROI at each iteration. In other words, the ROI may increase in size (more specifically, vertical height) by two pixel rows. The ROI may be an area constituted by an equal number of pixel rows of the first image (starting from the bottom-most pixel row) and the second image (starting from the top-most pixel row) at each iteration. The ROI of an image may be subjected to further processing in each iteration. The ROI at a final iteration may be the area constituted by each of the plurality of pixel rows of the first image and each of the plurality of pixel rows of the second image.

Thereafter, the computing device 102 may calculate an intensity difference between each of a plurality of first image ROI pixels and each of a corresponding plurality of second image ROI pixels. The plurality of first image ROI pixels may be pixels in an ROI section that are a part of the first image. In other words, the plurality of first image ROI pixels may include the pixel rows of the first image that form the ROI in each iteration. Similarly, the plurality of second image ROI pixels may be pixels in an ROI section that are a part of the second image. Based on the calculated intensity difference, the computing device 102 may determine a mean intensity difference for the ROI. Further, based on the mean intensity, the computing device 102 may identify an overlapping ROI from a plurality of ROIs. The plurality of ROIs include the ROI created and processed at each iteration. The overlapping ROI may include a minimum mean intensity difference amongst the plurality of ROIs.

In some embodiments, the computing device 102 may include one or more processors 104 and a memory 106. The memory 106 may store instructions that, when executed by the one or more processors 104, cause the one or more processors 104 to stitch anatomical images, in accordance with aspects of the present disclosure. The memory 106 may also store various data (for example, input anatomical images, an intensity difference, a mean intensity difference, and the like) that may be captured, processed, and/or required by the computing device 102. The memory 106 may be a non-volatile memory (e.g., flash memory, Read Only Memory (ROM), Programmable ROM (PROM), Erasable PROM (EPROM), Electrically EPROM (EEPROM) memory, etc.) or a volatile memory (e.g., Dynamic Random Access Memory (DRAM), Static Random-Access memory (SRAM), etc.).

The system 100 may further include a display 108. The system 100 may interact with a user via a user interface 110 accessible via the display 108. The system 100 may also include one or more external devices 112. In some embodiments, the computing device 102 may interact with the one or more external devices 112 over a communication network 114 for sending or receiving various data. The external devices 112 may include, but may not be limited to, a remote server, a digital device, or another computing system.

Referring now to FIG. 2, a functional block diagram of the memory 106 of the computing device 102 configured to stitch anatomical images is illustrated, in accordance with some embodiments. FIG. 2 is explained in conjunction with FIG. 1. The memory 106 may include a pre-processing module 202, an overlapping ROI identification module 204, a shift management module 206, a blending module 208, and a stitching module 210.

The pre-processing module 202 may receive a first image 212 and a second image 214. In an embodiment, the first image 212 and the second image 214 may be provided as an input by a user via a Graphical User Interface (GUI) rendered on a user device. The first image 212 may correspond to a first anatomical region and the second image 214 may correspond to a second anatomical region. The first image 212 and the second image 214 may be anatomical images captured by a medical imaging device (for example, an MRI scanner, a CT scanner, an X-ray machine, or the like). It may be noted that the first anatomical region and the second anatomical region may be in continuity in the body. By way of an example, the first anatomical region and the second anatomical region may be the cervical region (including C1 to C7 vertebrae) and the thoracic region (including T1 to T12 vertebrae), respectively, of the spine region. By way of another example, the first anatomical region and the second anatomical region may be the thoracic region (including T1 to T12 vertebrae) and the lumbar region (including L1 to L5 vertebrae), respectively, of the spine region. The first image 212 may include an overlapping region with the second image 214. It should be noted that the overlapping region may be the region common between the first image 212 and the second image 214. For example, when the first image 212 corresponds to the cervical region and the second image 214 corresponds to the thoracic region, the first image 212 may include the C1 to C7 vertebrae and the T1 to T3 vertebrae. The T1-T3 vertebrae may be observed in a bottom portion of the first image 212. Additionally, the second image 214 may include the C5 to C7 vertebrae and the T1-T12 vertebrae. The C5-C7 vertebrae may be observed in a top portion of the second image 214. This may be because while capturing an image, the medical imaging device may be focused on a corresponding anatomical region which may make up a majority portion of the captured image. However, some parts of anatomical regions adjacent to the corresponding anatomical region may also be captured in the image.

In an embodiment, the first image 212 and the second image 214 may be marker-free images. In other words, a reference marker may be absent in each of the first image 212 and the second image 214. The reference marker is manually applied while image capturing, adding to complexity of medical imaging process. The reference marker may then be used to identify an overlapping region in anatomical images, facilitating in stitching the anatomical images. However, a shadow of the reference marker may add noise to diagnostically relevant information in the stitched image. Thus, the received first image 212 and the second image 214 may or may not be marker-free images.

Further, the pre-processing module 202 may pre-process the first image 212 and the second image 214 using one or more pre-processing techniques (for example, image clipping, noise removing, thresholding, histogram equalization, geometric transformation, etc.). The pre-processing module 202 may calculate an image mean intensity (i.e., an average intensity across all pixels of an image) of each of the first image 212 and the second image 214. Further, the pre-processing module 202 may determine a maximum image mean intensity amongst the image mean intensities of the first image 212 and the second image 214. Based on the maximum image mean intensity, the pre-processing module 202 may calculate a contrast factor of each of the first image 212 and the second image 214. The contrast factor may be a ratio of the maximum image mean intensity and the image mean intensity. In some embodiments, the pre-processing module 202 may receive additional anatomical images (i.e., anatomical images in addition the first image 212 and the second image 214). In such embodiments, the maximum image mean intensity may be determined amongst a plurality of image mean intensities corresponding to the additional anatomical images, the first image 212, and the second image 214.

Further, the pre-processing module 202 may adjust a contrast of each of the first image 212 and the second image 214 based on the contrast factor in order to normalize the contrast of the first image 212 and the second image 214. In other words, the pre-processing module 202 may attain uniformity of contrast between the first image 212 and the second image 214. In some embodiments, the pre-processing module 202 may also normalize brightness of the first image 212 and the second image 214 through a brightness factor calculated in a similar manner to the contrast factor. In some embodiments, the pre-processing module 202 may apply a histogram equalization technique to each of the first image 212 and the second image 214. The histogram equalization may enhance the contrast of each of the first image 212 and the second image 214. As will be appreciated, a histogram of an image obtained through the histogram equalization technique may be evenly distributed over the x-axis and may provide a high-contrast effect to the image. Upon applying the one or more pre-processing techniques, the pre-processing module 202 may send the first image 212 and the second image 214 to the overlapping ROI identification module 204.

Further, for each each of a plurality of pixel rows of the first image 212 selected from bottom to top, and for each of a plurality of pixel rows of the second image selected from top to bottom, the overlapping ROI identification module 204 may cumulatively create an ROI from a first image pixel row with a second image pixel row until the ROI may include each of the plurality of pixel rows of each of the first image 212 and the second image 214. A pixel row may be a row of a predefined pixel height (for example, 1 pixel, 2 pixels, etc.), extending horizontally from one edge of an image to an opposite edge. It should, therefore, be noted that the pixel row may include a plurality of pixels. Two pixel rows (one from each of the first image 212 and the second image 214 may be added to the ROI at each iteration. In other words, the ROI may increase in size (more specifically, vertical height) by two pixel rows. The ROI at a final iteration may be the area constituted by each of the plurality of pixel rows of the first image 212 and each of the plurality of pixel rows of the second image 214.

The overlapping ROI identification module 204 may further calculate an intensity difference between each of a plurality of first image ROI pixels and each of a corresponding plurality of second image ROI pixels. The plurality of first image ROI pixels may be pixels in an ROI section that are a part of the first image 212. In other words, the plurality of first image ROI pixels may include pixels from the pixel rows of the first image 212 that form the ROI in each iteration. Similarly, the plurality of second image ROI pixels may be pixels in an ROI section that are a part of the second image 214. The overlapping ROI identification module 204 may calculate an intensity of a first pixel of the plurality of first image ROI pixels and an intensity of a corresponding first pixel of the plurality of second image ROI pixels. To elaborate, if the first pixel of the plurality of first image ROI pixels is a pixel of a top left corner of the ROI section of the first image 212, then the corresponding first pixel of the plurality of second image ROI pixels is a pixel of a top left corner of the ROI section of the second image 214.

Based on the calculated intensity difference, the overlapping ROI identification module 204 may determine a mean intensity difference for the ROI. The mean intensity difference may be a mean of intensity differences between the plurality of first image ROI pixels and the corresponding plurality of second image ROI pixels. Finally, the overlapping ROI identification module 204 may identify an overlapping ROI from a plurality of ROIs based on the mean intensity difference. The overlapping ROI may be an ROI with a minimum mean intensity difference amongst mean intensity differences of the plurality of ROIs. It should be noted that a plurality of anatomical images (two or more anatomical images) may be input to the computing device 102. However, the overlapping ROI identification module 204 may identify the overlapping ROI between two anatomical images at a time. Further, the overlapping ROI identification module 204 may send the first image 212, the second image 214, and overlapping ROI information to the shift management module 206.

The shift management module 206 may identify a horizontal shift between the first image 212 and the second image 214. The shift management module 206 may align the first image 212 with the second image 214 to obtain an initial alignment. The initial alignment may include the overlapping ROI of the first image 212 superimposed upon the overlapping ROI of the second image 214. Additionally, a plurality of pixel columns of the first image 212 may be aligned with a corresponding plurality of pixel columns of the second image 214 in the initial alignment. In other words, a first column and a last column of the plurality of pixel columns of the first image 212 may be aligned with a first column and a last column of the plurality of pixel columns of the second image 214, respectively.

Iteratively for each horizontal direction, the shift management module 206 may shift the overlapping ROI of the second image 214 by a pixel column towards the horizontal direction from the initial alignment to obtain a shifted alignment until the second image 214 is completely unaligned with the first image 212. A pixel column may be a column of a predefined pixel width (for example, 1 pixel, 2 pixels, etc.), extending vertically from one edge of an image to an opposite edge. It should, therefore, be noted that the pixel column may include a plurality of pixels. The shifted alignment may include one or more outlying pixel columns. In other words, the shift management module 206 may horizontally move the overlapping ROI of the second image 214 at each iteration by one pixel column from the superimposed initial alignment. The shifted alignment may be obtained for each side (left and right), until the final iteration (i.e., when none of a plurality of pixel columns of the second image 214 is aligned with a plurality of pixel columns of the first image 212).

Further, for each iteration, the shift management module 206 may generate a difference image from the shifted alignment. The one or more outlying pixel columns may be excluded in the difference image. The difference image may include pixels of the overlapping ROI of the first image 212 and the second image 214 which are superimposed in the shifted alignment. In other words, a difference image may be obtained from a difference between the superimposed overlapping ROI of the initial alignment and the superimposed overlapping ROI of the shifted alignment. Further, the shift management module 206 may calculate a mean intensity of the difference image. Further, for each horizontal direction (i.e., left direction and right direction), the shift management module 206 may determine a direction-wide minimum mean intensity from a plurality of mean intensities corresponding to a plurality of difference images obtained from the horizontal direction. Further, the shift management module 206 may determine an overall minimum mean intensity from each of direction-wide minimum mean intensities. The shifted alignment corresponding to the overall minimum mean intensity may be selected as an optimal alignment of the first image 212 and the second image 214. A number of plurality of pixel columns shifted to obtain the optimal shifted alignment may be the horizontal shift between the first image 212 and the second image 214. Based on the identified horizontal shift, the shift management module 206 may realign the second image 214 with the first image 212 to obtain a realigned alignment. The outlying pixel columns of the realigned alignment may be removed. The overlapping ROIs of the first image 212 and the second image 214 may be superimposed on each other in the realigned alignment. Further, the shift management module 206 may send the first image 212, the second image 214, and realigned alignment information to the blending module 208.

The blending module 208 may blend the first image 212 with the second image 214 through a blending technique (for example, alpha blending technique). To blend the first image 212 with the second image 214, the blending module 208 may split the overlapping ROI from each of the first image 212 and the second image 214 into two (preferably, equal) parts, i.e., a first part and a second part. The first part and the second part are mutually exclusive. Further, the blending module 208 may send the first part and the second part of each of the first image 212 and the second image 214 to the stitching module 210. Further, the stitching module 210 may obtain the first part of the overlapping ROI from the first image 212 and the second part of the overlapping ROI from each of the first image and the second image 214.

The stitching module 210 may blend the second part of the first image 212 and the second part of the second image 214 through the blending technique to obtain a blended second part. Finally, the stitching module 210 may modify a joining pixel row of the blended second part and the first part based on an average of adjacent pixel rows to the joining pixel row to obtain a blended image. The joining pixel row may correspond to a joining edge of the first part and the blended second part. In an embodiment, more than one predefined number of joining pixel rows may be modified. Further, based on the blending, the stitching module 210 may stitch the first image 212 with the second image 214 to obtain a stitched image 216. The stitched image 216 may be a seamlessly blended image of the first image 212 and the second image 214. Further, the stitched image 216 (or the composed image) may be rendered via a GUI on the user device.

It should be noted that all such aforementioned modules 202-210 may be represented as a single module or a combination of different modules. Further, as will be appreciated by those skilled in the art, each of the modules 202-210 may reside, in whole or in parts, on one device or multiple devices in communication with each other. In some embodiments, each of the modules 202-210 may be implemented as dedicated hardware circuit comprising custom application-specific integrated circuit (ASIC) or gate arrays, off-the-shelf semiconductors such as logic chips, transistors, or other discrete components. Each of the modules 202-210 may also be implemented in a programmable hardware device such as a field programmable gate array (FPGA), programmable array logic, programmable logic device, and so forth. Alternatively, each of the modules 202-210 may be implemented in software for execution by various types of processors (e.g., processor 104). An identified module of executable code may, for instance, include one or more physical or logical blocks of computer instructions, which may, for instance, be organized as an object, procedure, function, or other construct. Nevertheless, the executables of an identified module or component need not be physically located together but may include disparate instructions stored in different locations which, when joined logically together, include the module and achieve the stated purpose of the module. Indeed, a module of executable code could be a single instruction, or many instructions, and may even be distributed over several different code segments, among different applications, and across several memory devices.

As will be appreciated by one skilled in the art, a variety of processes may be employed for stitching anatomical images. For example, the exemplary system 100 and the associated computing device 102 may stitch the anatomical images by the processes discussed herein. In particular, as will be appreciated by those of ordinary skill in the art, control logic and/or automated routines for performing the techniques and steps described herein may be implemented by the system 100 and the associated computing device 102 either by hardware, software, or combinations of hardware and software. For example, suitable code may be accessed and executed by the one or more processors on the system 100 to perform some or all of the techniques described herein. Similarly, application specific integrated circuits (ASICs) configured to perform some, or all of the processes described herein may be included in the one or more processors on the system 100.

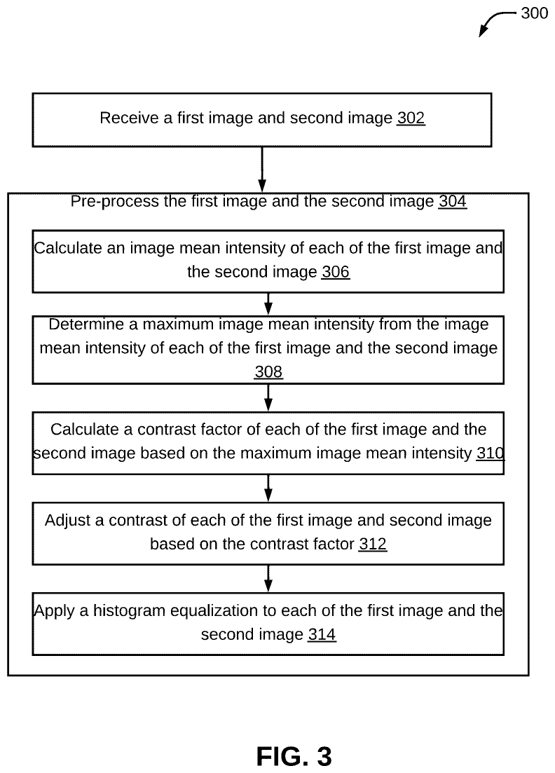

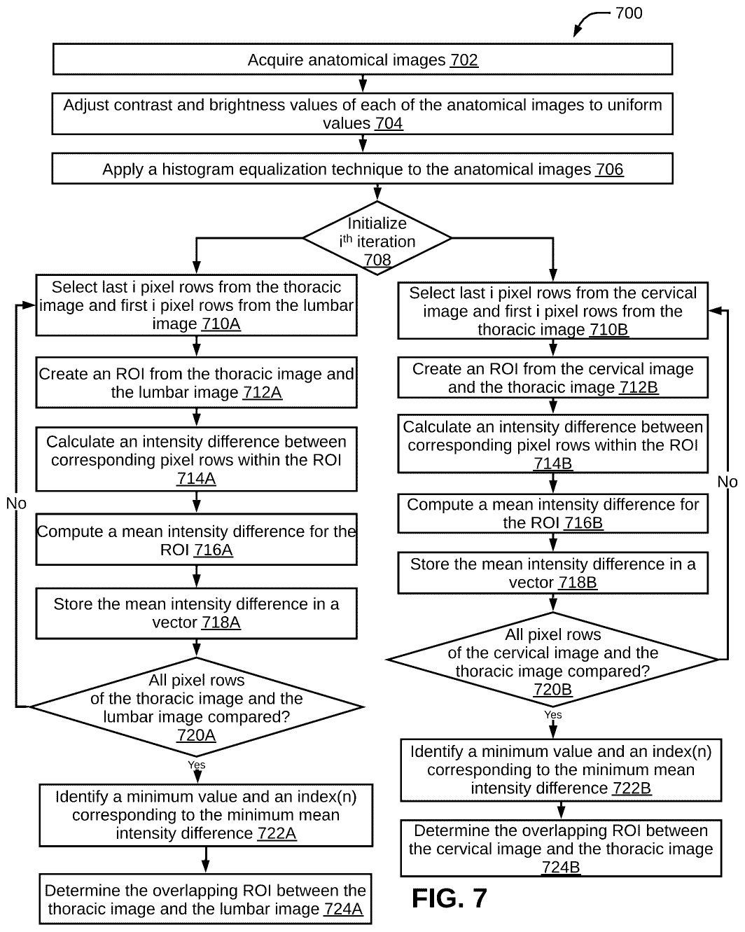

Referring now to FIG. 3, an exemplary process 300 for pre-processing a first image and a second image is illustrated, in accordance with some embodiments. FIG. 7 is explained in conjunction with FIGS. 1 and 2. The process 300 may be implemented by the computing device 102 of the system 100. The process 300 may include receiving a first image (for example, the first image 212) and a second image (for example, the second image 214), at step 302. The first image may correspond to a first anatomical region and the second image may correspond to a second anatomical region. The first image may include an overlapping region with the second image.

Further, the process 300 may include pre-processing the first image and the second image, at step 304. The step 304 of the process 300 may include calculating an image mean intensity of each of the first image and the second image, at step 306. Further, the step 304 of the process 300 may include determining a maximum image mean intensity from the image mean intensity of each of the first image and the second image, at step 308. Further, the step 304 of the process 300 may include calculating a contrast factor of each of the first image and the second image based on the maximum image mean intensity, at step 310.

Further, the step 304 of the process 300 may include adjusting a contrast of each of the first image and the second image based on the contrast factor, at step 312. Further, the step 304 of the process 300 may include applying a histogram equalization to each of the first image and the second image, at step 314. By way of an example, the pre-processing module 202 may calculate the image intensity of each of the first image 212 and the second image 214. Further, the pre-processing module 202 may determine the maximum image mean intensity from the image mean intensity of each of the first image 212 and the second image 214. Based on the maximum image mean intensity, the pre-processing module 202 may calculate the contrast factor of each of the first image 212 and the second image 214 and may further adjust the contrast of each of the first image 212 and the second image 214. Additionally, the pre-processing module 202 may apply the histogram equalization to each of the first image 212 and the second image 214.

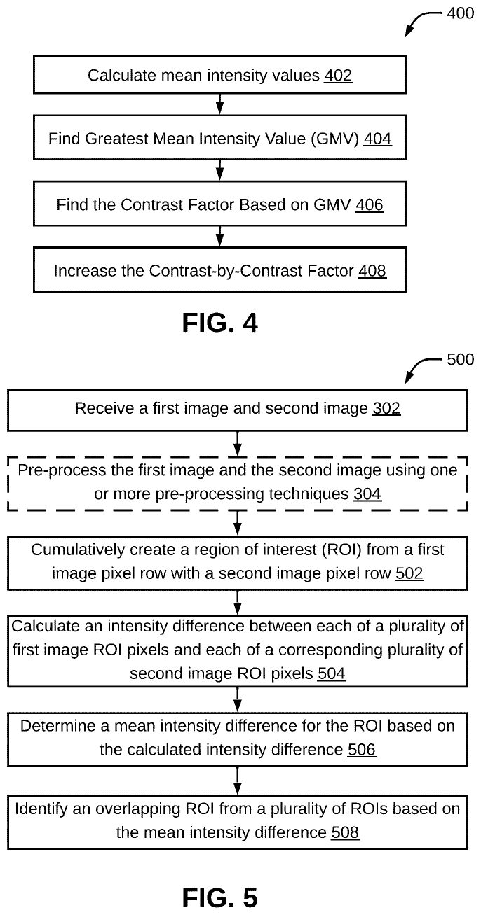

Referring now to FIG. 4, a detailed exemplary process 400 to pre-process anatomical images (for example, the cervical image, the thoracic image, and the lumbar image) is illustrated, in accordance with an embodiment. FIG. 4 is explained in conjunction with FIGS. 1, 2, and 3. The process 400 may be implemented by the computing device 102 of the system 100. The process 400 may include calculating a mean intensity value of each of the anatomical images, at step 402. By way of an example, the pre-processing module 202 may receive a cervical image, a thoracic image, and a lumbar image corresponding to the cervical region, the thoracic region, and the lumbar region, respectively. Further, the pre-processing module 202 may calculate the mean intensity value of each of the cervical image, the thoracic image, and the lumbar image.

Further, the process 400 may include calculating a Greatest Mean Intensity value (GMV) amongst the mean intensity value of each of the anatomical images, at step 404. In continuation of the example above, the GMV may be the maximum (max) mean intensity value among the mean intensity value of the cervical image, thoracic image, and the lumbar image as represented in equation (1) below.

$$GMV = max ( mean intensity of cervical image , mean intensity of thoracic image , mean intensity of lumbar image ) \qquad ( 1 )$$

Further, the process 400 may include finding a contrast factor based on the calculated GMV, at step 406. The contrast factor for an anatomical image may be a ratio between the GMV and the mean intensity of the anatomical image. In continuation of the example above, the contrast factor for each of the cervical image, thoracic image, and the lumbar image may calculated through equations (2), (3), and (4), respectively.

$$Contrast factor ( cervical image ) = GMV / mean intensity ( cervical image ) \qquad ( 2 )$$

$$Contrast factor ( thoracic image ) = GMV / mean intensity ( thoracic image ) \qquad ( 3 )$$

$$\begin{matrix}Contrast factor ( lumbar image ) = GMV / mean intensity ( lumbar image ) & ( 4 )\end{matrix}$$

Finally, the process 400 may include increasing the contrast of each of the anatomical images based on the contrast factor, at step 408.

Referring now to FIG. 5, an exemplary process 500 for identifying an overlapping ROI between anatomical images is depicted via a flowchart, in accordance with some embodiments. FIG. 5 is explained in conjunction with FIGS. 1, 2, and 3. The process 500 may be implemented by the computing device 102 of the system 100. The process 500 may include receiving a first image (for example, the first image 212) and a second image (for example, the second image 214), at step 302. In some embodiments, the process 500 may include pre-processing the first image and the second image using the one or more pre-processing techniques (for example, contrast normalization and histogram equalization), at step 304. This has already been explained in detail in conjunction with FIGS. 3 and 4. Further, for each of a plurality of pixel rows of the first image selected from bottom to top, and for each of a plurality of pixel rows of the second image selected from top to bottom, the process 500 may include cumulatively creating a ROI from a first image pixel row with a second image pixel row until the ROI may include each of the plurality of pixel rows of each of the first image and the second image, at step 502.

By way of an example, the overlapping ROI identification module 204 may receive the first image 212 and the second image 214 from the pre-processing module 202. Further, the overlapping ROI identification module 204 may cumulatively create an ROI from a first image pixel row with a second image pixel row until the ROI may include each of the plurality of pixel rows of each of the first image 212 and the second image 214. It is worth noting that at an initial iteration, the ROI may include one pixel row each from the first image 212 and the second image 214. The pixel row from the first image 212 may be the bottom-most pixel row of the first image 212 and the pixel row from the second image 214 may be the top-most pixel row of the second image 214. At a next iteration, the ROI may include two pixel rows each from the first image 212 and the second image 214. The two pixel rows from the first image 212 may be the bottom-most pixel row of the first image 212 and a second bottom-most pixel row (i.e., the pixel row immediately above the bottom-most pixel row) of the first image 212. Similarly, the two pixel rows from the second image 214 may be the top-most pixel row of the second image 214 and a second top-most pixel row of the second image 214. Also, at a final iteration, the ROI may include each of the plurality of pixel rows from each of the first image 212 and the second image 214.

Further, the process 500 may include calculating an intensity difference between each of a plurality of first image ROI pixels and each of a corresponding plurality of second image ROI pixels, at step 504. Further, the process 500 may include determining a mean intensity difference for the ROI based on the calculated intensity difference, at step 506. Finally, the process 500 may include identifying an overlapping ROI from a plurality of ROIs based on the mean intensity difference, at step 508. It should be noted that the overlapping ROI may include a minimum mean intensity difference.

In continuation of the example above, the overlapping ROI identification module 204 may calculate an intensity difference between each of the plurality of first image ROI pixels and each of a corresponding plurality of second image ROI pixels. Location of a first image ROI pixel in the ROI section of the first image 212 may be same as location of a corresponding second image ROI pixel in the ROI section of the second image 214. For example, if a pixel of the plurality of first image ROI pixels is a pixel at a top left corner of the ROI section of the first image 212, then the corresponding pixel of the plurality of second image ROI pixels is a pixel at a top left corner of the ROI section of the second image 214. Based on the calculated intensity difference, the overlapping ROI identification module 204 may determine a mean intensity difference for the ROI. Finally, based on the mean intensity difference, the overlapping ROI identification module 204 may identify an overlapping ROI from a plurality of ROIs. The overlapping ROI may include a minimum mean intensity difference. It should be noted that the identified overlapping ROI may be an estimate of the overlapping region between the first image 212 and the second image 214.

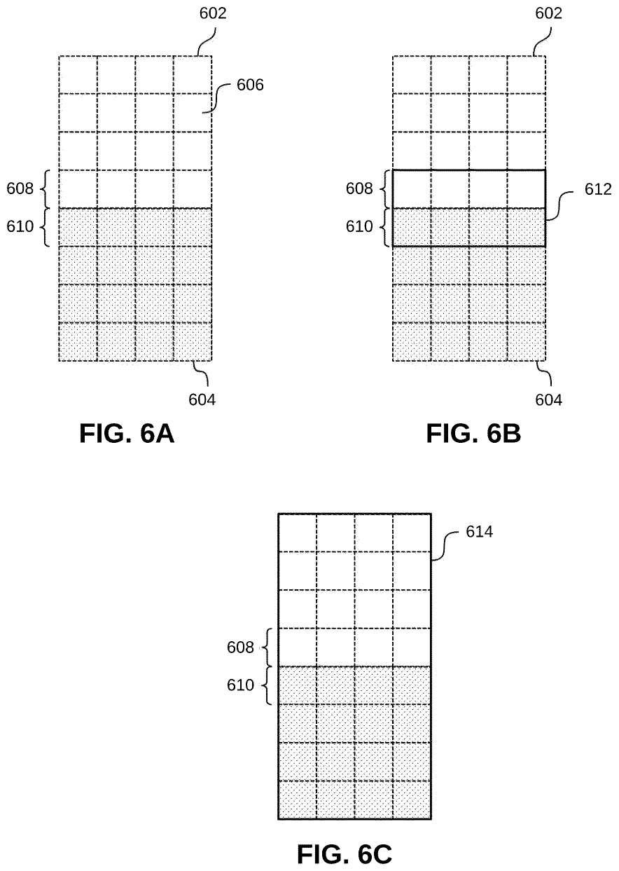

Referring now to FIGS. 6A-C, schematic diagrams of cumulative creation of ROIs from anatomical images are illustrated, in accordance with some embodiments. FIGS. 6A-C are illustrated in conjunction with FIGS. 2 and 5. In FIG. 6A, a first image 602 (analogous to the first image 212) is vertically aligned with a second image 604 (analogous to the second image 214). Upon pre-processing by the pre-processing module 202, the first image 602 and the second image 604 are received by the overlapping module 204. Each of the first image 602 and the second image 604 may include a plurality of pixels (for example, a pixel 606) arranged in a plurality of pixel rows and a plurality of pixel columns. As will be appreciated, an image is a matrix of a plurality of pixels. By way of an example, the first image 602 may include a first pixel row 608 and the second image 604 may include a first pixel row 610. It should be noted that the first pixel row 608 is the bottom-most pixel row of the first image 602 and the first pixel row 610 is the top-most pixel row of the second image 604. Further, each of the first pixel row 608 and the first pixel row 610 includes 4 pixels.

In FIG. 6B, at an initial iteration, the overlapping ROI identification module 204 may create a first ROI 612 including the first pixel row 608 and the first pixel row 610. Further, the overlapping ROI identification module 204 may calculate an intensity difference between each of a plurality of ROI pixels in the first pixel row 608 and each of a corresponding plurality of ROI pixels in the first pixel row 610. Further, the overlapping ROI identification module 204 may determine a mean intensity difference for the first ROI 612 based on the calculated intensity difference.

In FIG. 6C, at a final iteration, the overlapping ROI identification module 204 may create a last ROI 614 including each of the plurality of pixel rows of the first image 602 and each of the plurality of pixel rows of the second image 604. Further, the overlapping ROI identification module 204 may calculate an intensity difference between each of the plurality of pixels in the first image 602 and each of the corresponding plurality of pixels in the second image 604. Further, the overlapping ROI identification module 204 may determine a mean intensity difference for the last ROI 614 based on the calculated intensity difference.

Further, the overlapping ROI identification module 204 may identify an overlapping ROI from a plurality of ROIs obtained and processed at a plurality of iterations (i.e., from the initial iteration to the final iteration). Thus, the plurality of ROIs may include the first ROI 612, the last ROI 614, and any number of ROIs obtained through the plurality of iterations in between the initial iteration and the final iteration.

Referring now to FIG. 7, a detailed exemplary process 700 for identifying an overlapping ROI is depicted via a flowchart, in accordance with an embodiment. FIG. 7 is explained in conjunction with FIGS. 1, 2, 3, and 5. The process 700 may be implemented by the computing device 102 of the system 100. The process 700 may include acquiring, by a pre-processing module (such as the pre-processing module 202), anatomical images (for example, the cervical image, the thoracic image, and the lumbar image), at step 702. The anatomical images may be analogous to the first image 212 and the second image 214. In an embodiment, the anatomical images may be MRI scan images. The anatomical images may be in a Digital Imaging and Communications in Medicine (DICOM) format. The anatomical images may be in one of a sagittal view or a coronal view. It should be noted that at a time, the anatomical images with same view (i.e., either sagittal or coronal) may be acquired and received for further processing. Further, the process 700 may include adjusting, by the pre-processing module, contrast and brightness values of each of the anatomical images to uniform values, at step 704. This has already been explained in detail in conjunction with FIGS. 3 and 4.

Further, the process 700 may include applying, by the pre-processing module, a histogram equalization technique to the anatomical images, at step 706. This has already been explained in detail in conjunction with FIGS. 3 and 4. The histogram equalization technique may use a formula represented in equation (5) to obtain an equalized value of intensity levels for an image.

$$Equalized value ( i ) = round ( \frac{cdf ( i ) - {cdf}_{min}}{1 - {cdf}_{min}} X ( L - 1 ) ) \qquad ( 5 )$$

Where,
cdf(i) is a cumulative distribution function,
cdfₘᵢₙ is a minimum non-zero CDF value, and
L is a number of intensity levels (defined at 256 for an 8-bit image)

Further, the process 700 may include initializing, by an overlapping ROI identification module (such as the overlapping ROI identification module 204), an i^{th} iteration of a plurality of iterations for two anatomical images, at step 708. A number (i.e., count) of the plurality of iterations may be equal to a number of the pixel rows in each of the anatomical images. In other words, i may be in a range of 1 to the number of the pixel rows in each of the anatomical images. While in some cases more than two anatomical images may be required to obtain a composite image, the overlapping ROI identification module may process two continuous anatomical images at a time. For ease of explanation, the two continuous anatomical images may be a first image (analogous to the first image 212) and a second image (analogous to the second image 214). By way of an example, three anatomical images may be received, i.e., the cervical image, the thoracic image, and the lumbar image. Each two continuous anatomical images from the three anatomical images may be processed at a time. Thus, the thoracic image and the lumbar image may be processed through a sub-process 700A. Additionally, the cervical image and the thoracic image may be processed through a sub-process 700B.

Further, at the i^{th} iteration, the sub-process 700A may include selecting last i pixel rows (i.e., i pixel rows selected from the bottom) from the thoracic image and first i pixel rows (i.e., i pixel rows selected from the top) from the lumbar image, at step 710A. Thereafter, the sub-process 700A may include creating an ROI from the thoracic image and the lumbar image, at step 712A. Further, the sub-process 700A may include calculating an intensity difference between corresponding pixel rows within the ROI, at step 714A. The overlapping identification module 204 may perform a pixel row-wise comparison of pixel intensities between the thoracic image and the lumbar image. Initial comparison may include comparing a last pixel row of the thoracic image and a first pixel row of the lumbar image to create a ROI. Further, the sub-process 700A may include computing a mean intensity difference for the ROI, at step 716A. Further, the sub-process 700A may include storing the mean intensity difference in a vector, at step 718A. Further, at step 720A of the sub-process 700A, a check may be performed to determine whether each of the plurality of pixel rows of the thoracic image and the lumbar image are compared.

If each of the plurality of pixel rows are not compared, the sub-process 700A may return to the step 710A (“No” path). Accordingly, the steps 710A to 720A may be repeated until each of the plurality of pixel rows of the thoracic image and the lumbar image are compared.

If each of the plurality of pixel rows of the thoracic image and the lumbar image are compared, the sub-process 700A may proceed to step 722A. The sub-process 700A may include identifying, by the overlapping identification module, a minimum value and an index(n) corresponding to the minimum mean intensity difference, at step 722A. The index(n) may correspond to an index number of a bottom-most ROI pixel row of the lumbar image and a top-most ROI pixel row of the thoracic image of the ROI corresponding to the minimum mean intensity difference. The index(n) may be a location of pixel rows that constitute top and bottom edges of the ROI. Further, the sub-process 700A may include determining, by the overlapping identification module, the overlapping ROI between the thoracic image and the lumbar image, at step 724A. It should be noted that the overlapping ROI may be the ROI corresponding to the minimum mean intensity difference.

Similarly, the overlapping ROI identification module 204 may identify an overlapping region for a cervical image (analogous to the first image 212) and the thoracic image (analogous to the second image 214) through the sub-process 700B. The sub-process 700B may include steps 710B, 712B, 714B, 716B, 718B, 720B, 722B, and 724B. The steps 710B-724B are analogous to the steps 710A-724A, respectively.

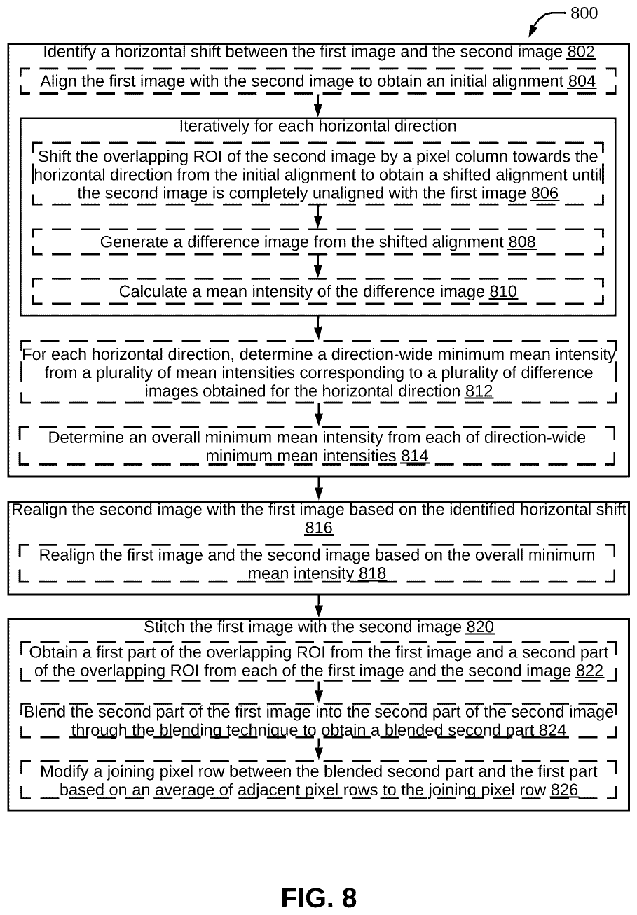

Referring now to FIG. 8, an exemplary process 800 for stitching anatomical images (for example, the first image 212 and the second image 214) is illustrated via a flowchart, in accordance with some embodiments. FIG. 8 is explained in conjunction with FIGS. 1, 2, and 5. The process 800 may be implemented by the computing device 102 of the system 100. It may be noted that the process 800 may be implemented in continuation with the process 500. Once the overlapping ROI is identified (as explained in detail in conjunction with FIG. 5), the process 800 may include identifying a horizontal shift between the first image (for example, the first image 212) and the second image (for example, the second image 214), at step 802.

The step 802 of the process 800 may include aligning the first image with the second image to obtain an initial alignment, at step 804. It may be noted that the initial alignment may include the overlapping ROI of the first image superimposed upon the overlapping ROI of the second image. Further, iteratively for each horizontal direction, the step 802 of the process 800 may include shifting the overlapping ROI of the second image by a pixel column towards the horizontal direction from the initial alignment to obtain a shifted alignment until the second image is completely unaligned with the first image, at step 806. It may be noted that the shifted alignment may include one or more outlying pixel columns. Further, iteratively for each horizontal direction, the step 802 of the process 800 may include generating a difference image from the shifted alignment, at step 808. It may be noted that the one or more outlying pixel columns may be excluded in the difference image. Further, iteratively for each horizontal direction, the step 802 of the process 800 may include calculating a mean intensity of the difference image, at step 810. Further, for each horizontal direction, the step 802 of the process 800 may include determining a direction-wide minimum mean intensity from a plurality of mean intensities corresponding to a plurality of difference images obtained for the horizontal direction, at step 812.

Further, the process 800 may include determining an overall minimum mean intensity from each of direction-wide minimum mean intensities, at step 816. By way of an example, upon identifying the overlapping ROI, the shift management module 206 may receive the first image 212, the second image 214, and the overlapping ROI information from the overlapping ROI identification module 204. Further, the shift management module 206 may superimpose the overlapping ROI of the first image 212 upon the overlapping ROI of the second image 214 to obtain the initial alignment. Further, the shift management module 206 may shift the overlapping ROI of the second image 214 towards one horizontal direction (i.e., left or right) by a pixel column at each iteration to obtain a shifted alignment until the second image 214 is completely unaligned with the first image 212. Thus, in a shifted alignment, one or more pixel columns of the second image 214 may be outlying (i.e., unaligned) with the first image 212 towards the side of the horizontal direction. Further, at each iteration, the shift management module 206 may generate a difference image from the shifted alignment. The difference image may include a plurality of pixels of the overlapping ROI of the second image 212 that are superimposed upon the overlapping ROI of the first image 212. In other words, the one or more outlying pixel columns are excluded in the difference image. Further, at each iteration, the shift management module 206 may calculate the mean intensity of the difference image. The iterations towards a horizontal direction may continue until the difference image includes a single pixel column. Upon completing the iterations in a horizontal direction, the shift management module 206 may determine a direction-wide minimum mean intensity in that horizontal direction. Further, the shift management module 206 may perform the iterations in an opposite horizontal direction and may determine a direction-wide minimum mean intensity in the opposite horizontal direction. Further, the shift management module 206 may determine an overall minimum mean intensity from among the two direction-wide minimum mean intensities.

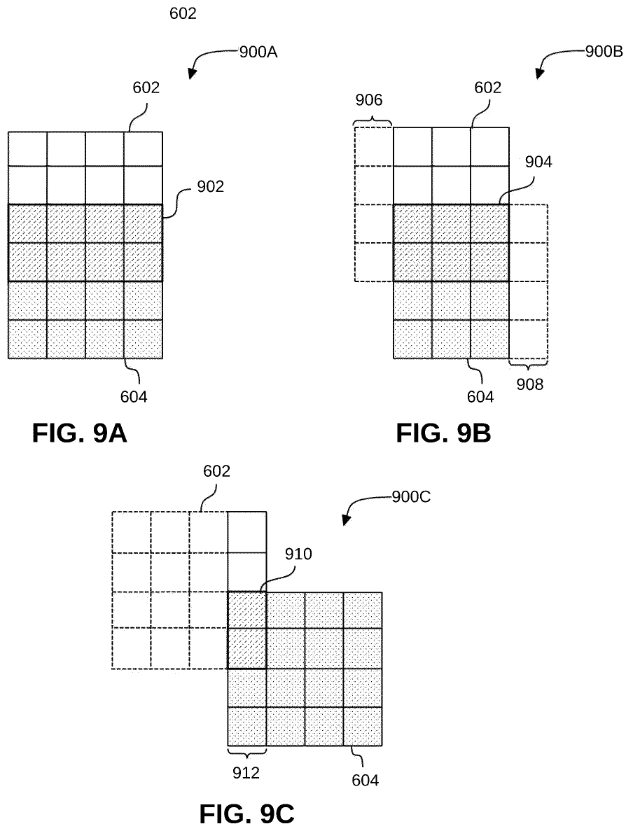

Referring now to FIGS. 9A-C, schematic diagrams of generation of difference images from shifted alignments of anatomical images are illustrated, in accordance with some embodiments. FIGS. 9A-C are explained in conjunction with FIGS. 2 and 8. In FIG. 9A, in an initial alignment 900A, the overlapping ROI of the second image 604 is superimposed upon the overlapping ROI of the first image 602. It should be noted that the overlapping ROI includes two pixel rows from each of the first image 602 and the second image 604. Further, the shift management module 206 may generate a first difference image 902 from the initial alignment 900A. The first difference image 902 may include pixels of the overlapping ROI of the second image 604 that are superimposed upon the overlapping ROI of the first image 602. Thus, the first difference image 902 may include each of the plurality of pixels in the overlapping ROI of the second image 604.

In FIG. 9B, in a first shifted alignment 900B, the overlapping region of the second image 604 is shifted by one pixel column in a rightward direction, at an initial iteration towards the rightward direction. Further, the shift management module 206 may generate a second difference image 904 from the first shifted alignment 900A. Additionally, the first shifted alignment 900B may include two outlying pixel columns, i.e., a pixel column 906 of the first image 602 and a pixel column 908 of the second image 604. The pixel column 906 may be left-most pixel column of the first image and the pixel column 908 may be right-most pixel column of the second image 604. The second difference image 904 may include pixels of the overlapping ROI of the second image 604 that are superimposed upon the overlapping ROI of the first image 602. Thus, ROI pixels of each of the pixel column 906 and the pixel column 908 may be excluded from the second difference image 904.

In FIG. 9C, through a last shifted alignment 9000, the shift management module 206 may generate a final difference image 910. The final difference image 910 may include pixels of the overlapping ROI of the second image 604 that are superimposed upon the overlapping ROI of the first image 602. Thus, final difference image 910 may include ROI pixels from a single pixel column, i.e., a pixel column 912 of the second image 604. Remaining of the ROI pixels from the second image 604 may be excluded from the final difference image 910.

It should be noted that the shift management module 206 may generate a difference image at each iteration. The first difference image 902 is generated from the initial alignment 900A (0^{th} iteration), the second difference image 904 is generated from the first shifted alignment 900B (initial iteration), the final difference image 910 is generated from the last shifted alignment 9000 (final iteration), and additional difference images may be generated in iterations between the first iteration and the final iteration. Further, at each iteration, the shift management module 206 may calculate a mean intensity of the difference image.

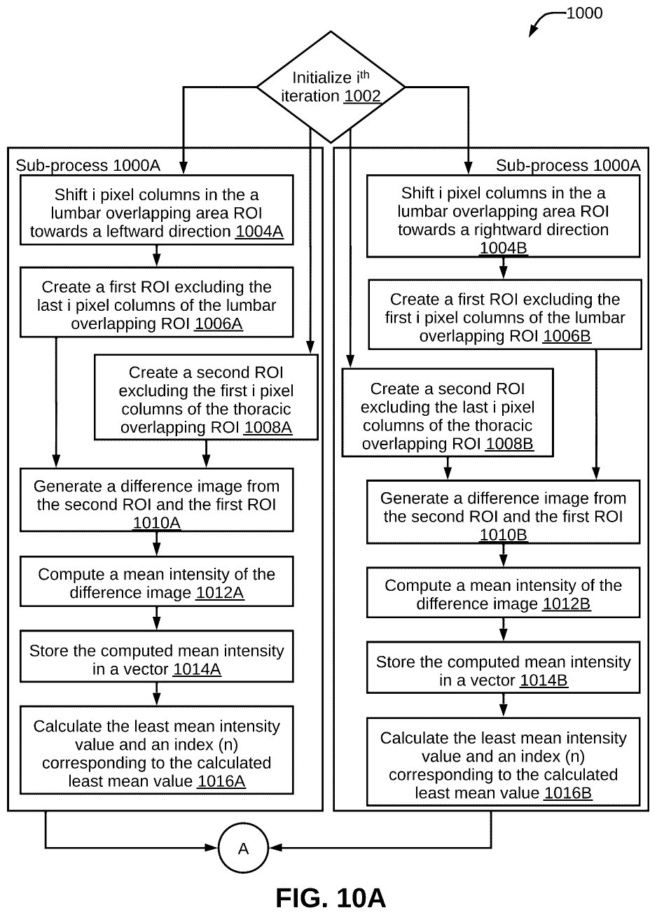

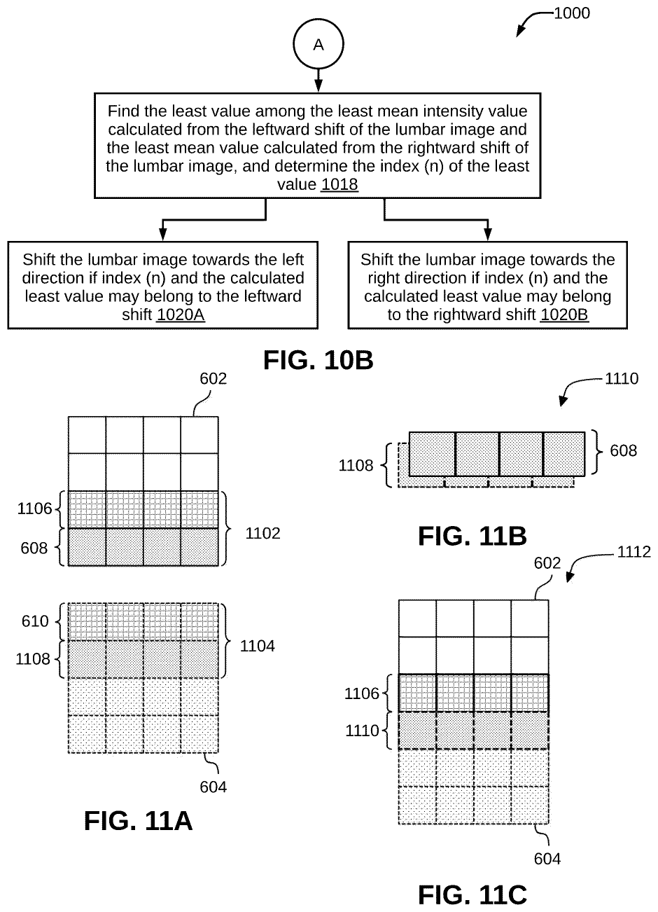

Referring now to FIGS. 10A and 10B, a detailed exemplary process 1000 for identifying horizontal shift between anatomical images (for example the first image 212 and the second image 214) is illustrated via a flowchart, in accordance with an embodiment. FIG. 10 is explained in conjunction with FIGS. 1, 2, 5, and 8. The process 1000 may be implemented by the computing device 102 of the system 100. Upon identifying the overlapping ROI between the first image and the second image, the process 1000 may include initializing, by a shift management module (such as the shift management module 206), an i^{th} iteration of a plurality of iterations for two anatomical images, at step 1002. A number (i.e., count) of the plurality of iterations may be equal to a number of the pixel columns in each of the anatomical images. In other words, i may be in a range of 1 to the number of the pixel columns in each of the anatomical images. The shift management module may process two continuous anatomical images in a horizontal direction at a time. By way of an example, two anatomical images may be received, i.e., the thoracic image and the lumbar image. The two continuous anatomical images may be processed in each horizontal direction (i.e., the leftward direction and the rightward direction). Thus, the thoracic image and the lumbar image may be processed in a leftward direction through a sub-process 1000A. Additionally, the thoracic image and the lumbar image may be processed in a rightward direction through a sub-process 1000B.

Further, at the i^{th} iteration, the sub-process 1000A may include shifting i pixel columns in a lumbar overlapping ROI towards the leftward direction, at step 1004A. At an initial iteration (0^{th} iteration), the lumbar overlapping ROI may be completely superimposed upon the thoracic overlapping ROI. It should be noted that a thoracic overlapping ROI may be kept unchanged while the lumbar overlapping ROI is shifted by one pixel column at each iteration. Further, the sub-process 1000A may include creating a first ROI excluding the last i pixel columns of the lumbar overlapping ROI, at step 1006A. Additionally, the sub-process 1000A may include creating a second ROI excluding the first i pixel columns of the thoracic overlapping ROI, at step 1008A. Upon creating the first ROI and the second ROI, the sub-process 1000A may include generating a difference image from the first ROI and the second ROI, at step 1010A. It should be noted that the difference image may be obtained by subtracting the second ROI from the first ROI.

Further, the sub-process 1000A may include computing a mean intensity of the difference image, at step 1012A. Further, the sub-process 1000A may include storing the computed mean intensity in a vector, at step 1014A. Thereafter, the sub-process 1000A may include calculating the least mean intensity value and an index (n) corresponding to the calculated least mean value, at step 1016A.

Similarly, the shift identification module 206 may calculate the least mean value of the lumbar image shifted in the rightward direction at each iteration through the sub-process 1000B that mya include steps 1004B, 1006B, 1008B, 1010B, 1012B, 1014B, and 1016B. The steps 1004B-1016B are analogous to the steps 1002A-1016A.

Further, the process 1000 may include finding, by the shift identification module, the least value among the least mean intensity value calculated from the leftward shift (i.e., minimum mean intensity in leftward direction) of the lumbar image and the least mean value calculated from the rightward shift (i.e., minimum mean intensity in rightward direction) of the lumbar image, and determining the index (n) of the least value, at step 1018. Finally, the process 1000 may include shifting, by the shift management module, the lumbar image towards the left direction if index (n) and the calculated least value may belong to the leftward shift, at step 1020A. Alternatively, the process 1000 may include shifting, by the shift management module, the lumbar image towards the right direction if the index (n) and the calculated mean value may belong to the rightward shift, at step 1020B.

Similarly, the shift management module may identify the horizontal shift between the cervical image (analogous to the first image 212) and the thoracic image (analogous to the second image 214) as explained in the step 1002-1020A-B.

Referring back to FIG. 8, the process 800 may include realigning the second image with the first image based on the identified horizontal shift, at step 816. Further, the step 816 of the process 800 may include realigning the first image and the second image based on the overall minimum mean intensity, at step 818. In continuation of the example, the shift management module 206 may identify a horizontal shift (i.e., a shifted alignment with the overall minimum mean intensity of the difference image) between the first image 212 and the second image 214. Further, the shift management module 206 in accordance with the shifted alignment to minimize the horizontal shift.

Further, upon realigning, the process 800 may include splitting the overlapping ROI of each of the first image and the second image into two parts. Further, the process 800 may include stitching the first image with the second image based on the blending, at step 820. The step 820 of the process 800 may include obtaining a first part of the overlapping ROI from the first image and a second part of the overlapping ROI from each of the first image and the second image, at step 822. It may be noted that the first part and the second part are mutually exclusive. Further, the step 820 of the process 800 may include blending the second part of the first image into the second part of the second image through the blending technique (for example, an alpha blending technique) to obtain a blended second part, at step 824. Further, the step 820 of the process 800 may include modifying a joining pixel row between the blended second part and the first part based on an average of adjacent pixel rows to the joining pixel row to obtain a stitched image (for example, the stitched image 216), at step 826.

In continuation of the example above, the blending module 208 may horizontally split the overlapping ROI of each of the first image 212 and the second image 214 into two parts (preferably of equal height). Further, the stitching module 210 may obtain a first part (i.e., an upper part) of the overlapping ROI from the first image 212 and a second part of the overlapping ROI from each of the first image 212 and the second image 214. Further, the stitching module 210 may blend the second part of the first image 212 into the second part of the second image 214 to obtain a blended second part. Additionally, the stitching module 210 may modify the joining pixel row of the blended second part and the first part to obtain the stitched image 216.

The stitched image (or the composed image) may be rendered via a GUI on a user device. By way of an example, a cervical image (analogous to the first image 212) may be stitched with the thoracic image (analogous to the second image 214) to obtain a first stitched image (analogous to the stitched image 216). Finally, the first stitched image (now analogous to the first image 212) may be stitched with the lumbar image (analogous to the second image 214) to obtain a final stitched image (analogous to the stitched image 216). Additionally, averaging in the transition area may be applied for a seamless stitch. Thus, any number of anatomical images may be stitched through the techniques described in the present disclosure.

Referring now to FIGS. 11A-C, schematic diagrams of blending of anatomical images are illustrated, in accordance with some embodiments. FIGS. 11A-C are explained in conjunction with FIGS. 2 and 8. In FIG. 11A, the first image 602 and the second image 604 are shown. The overlapping ROI identification module 206 may identify an overlapping ROI 1102 in the first image 602. Additionally, the overlapping ROI identification module 206 may identify an overlapping ROI 1104 in the second image 604. Each of the overlapping ROI 1102 and the overlapping ROI 1104 includes two pixel rows—the overlapping ROI 1102 includes the pixel row 608 and a pixel row 1106, and the overlapping ROI 1104 includes the pixel row 610 and a pixel row 1108.

In FIG. 11B, an overlapping ROI assembly 1110 is shown. The blending module 208 may horizontally split each of the overlapping ROI 1102 and the overlapping ROI 1104 into two parts, i.e., two pixel rows. Further, the stitching module 210 may obtain a first part (i.e., upper part) from the first image 602 and a second part (i.e., lower part) from each of the first image 602 and the second image 604. The overlapping ROI assembly 1110 may include the pixel row 608 superimposed upon the pixel row 1108. Further, the stitching module 210 may blend the pixel row 608 and the pixel row 1108 through a blending technique to obtain a blended second part (i.e., the overlapping ROI assembly 1110).

In FIG. 11C, a blended image 1112 is shown. The stitching module 210 may generate the blended image 1112 by blending (seamlessly joining) the first image 602 and the second image 604 from the pixel row 1106 (i.e., the first part of the overlapping ROI 1102 of the first image 602) and the overlapping ROI assembly 1110 (i.e., the blended second part), through a blending technique (for example, alpha blending).

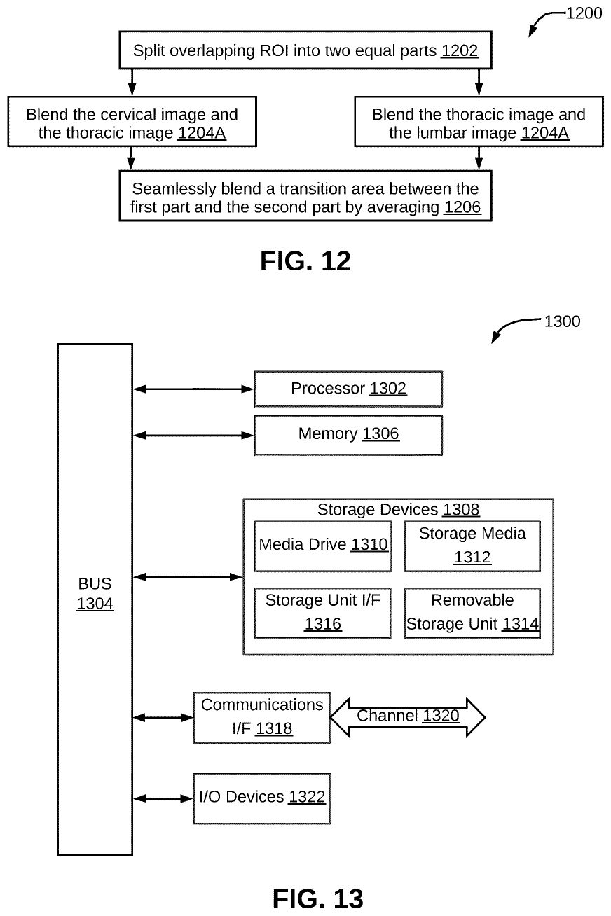

Referring now to FIG. 12, a detailed exemplary process 1200 for blending anatomical images (for example, the first image 212 and the second image 214) is illustrated via a flowchart, in accordance with an embodiments. FIG. 12 is explained in conjunction with FIGS. 1, 2, and 8. The process 1200 may be implemented by the computing device 102 of the system 100. The process 1200 may include splitting, by a blending module (such as the blending module 208), the identified overlapping ROI into two equal parts, at step 1202.

The process 1200 may include blending, by the blending module, a first part of the overlapping ROI of the cervical image (analogous to the first image 212) with a second part of the overlapping ROI of the thoracic image (analogous to the second image 214), at step 1204A. In an embodiment, the blending technique used may be an alpha blending technique with a predefined alpha factor value (e.g., 0.6). The alpha-blending technique may be used to blend the overlapping ROI. The alpha-blending technique may ensure a smooth transition between the cervical image, the thoracic image and the lumbar image. Additionally, the alpha-blending technique may minimize visible seams or discrepancies. Similarly, the process 1200 may include blending, by the stitching module, the first part of the overlapping ROI of the thoracic image (analogous to the first image 212) with the second part of the overlapping ROI of the lumbar image (analogous to the second image 214), at step 1204B.

Finally, the process 1200 may include seamlessly blending, by the blending module, a transition area between the first part and the second part by averaging, at step 1206. It should be noted that a center pixel row of the blended image may be replaced by an average of a previous pixel row and a next pixel row to obtain the seamlessly blended image.

As will be also appreciated, the above-described techniques may take the form of computer or controller implemented processes and apparatuses for practicing those processes. The disclosure can also be embodied in the form of computer program code containing instructions embodied in tangible media, such as floppy diskettes, solid state drives, CD-ROMs, hard drives, or any other computer-readable storage medium, wherein, when the computer program code is loaded into and executed by a computer or controller, the computer becomes an apparatus for practicing the invention. The disclosure may also be embodied in the form of computer program code or signal, for example, whether stored in a storage medium, loaded into and/or executed by a computer or controller, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

The disclosed methods and systems may be implemented on a conventional or a general-purpose computer system, such as a personal computer (PC) or server computer. Referring now to FIG. 13, an exemplary computing system 1300 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 1300 may represent, for example, a user device such as a desktop, a laptop, a mobile phone, personal entertainment device, DVR, and so on, or any other type of special or general-purpose computing device as may be desirable or appropriate for a given application or environment. The computing system 1300 may include one or more processors, such as a processor 1302 that may be implemented using a general or special purpose processing engine such as, for example, a microprocessor, microcontroller or other control logic. In this example, the processor 1302 is connected to a bus 1304 or other communication medium. In some embodiments, the processor 1302 may be an Artificial Intelligence (AI) processor, which may be implemented as a Tensor Processing Unit (TPU), or a graphical processor unit, or a custom programmable solution Field-Programmable Gate Array (FPGA).

The computing system 1300 may also include a memory 1306 (main memory), for example, Random Access Memory (RAM) or other dynamic memory, for storing information and instructions to be executed by the processor 1302. The memory 1306 also may be used for storing temporary variables or other intermediate information during execution of instructions to be executed by the processor 1302. The computing system 1300 may likewise include a read only memory (“ROM”) or other static storage device coupled to bus 1304 for storing static information and instructions for the processor 1302.

The computing system 1300 may also include a storage devices 1308, which may include, for example, a media drive 1310 and a removable storage interface. The media drive 1310 may include a drive or other mechanism to support fixed or removable storage media, such as a hard disk drive, a floppy disk drive, a magnetic tape drive, an SD card port, a USB port, a micro USB, an optical disk drive, a CD or DVD drive (R or RW), or other removable or fixed media drive. A storage media 1312 may include, for example, a hard disk, magnetic tape, flash drive, or other fixed or removable medium that is read by and written to by the media drive 1310. As these examples illustrate, the storage media 1312 may include a computer-readable storage medium having stored therein particular computer software or data.

In alternative embodiments, the storage devices 1308 may include other similar instrumentalities for allowing computer programs or other instructions or data to be loaded into the computing system 1300. Such instrumentalities may include, for example, a removable storage unit 1314 and a storage unit interface 1316, such as a program cartridge and cartridge interface, a removable memory (for example, a flash memory or other removable memory module) and memory slot, and other removable storage units and interfaces that allow software and data to be transferred from the removable storage unit 1314 to the computing system 1300.

The computing system 1300 may also include a communications interface 1318. The communications interface 1318 may be used to allow software and data to be transferred between the computing system 1300 and external devices. Examples of the communications interface 1318 may include a network interface (such as an Ethernet or other NIC card), a communications port (such as for example, a USB port, a micro USB port), Near field Communication (NFC), etc. Software and data transferred via the communications interface 1318 are in the form of signals which may be electronic, electromagnetic, optical, or other signals capable of being received by the communications interface 1318. These signals are provided to the communications interface 1318 via a channel 1320. The channel 1320 may carry signals and may be implemented using a wireless medium, wire or cable, fiber optics, or other communications medium. Some examples of the channel 1320 may include a phone line, a cellular phone link, an RF link, a Bluetooth link, a network interface, a local or wide area network, and other communications channels.

The computing system 1300 may further include Input/Output (I/O) devices 1322. Examples may include, but are not limited to a display, keypad, microphone, audio speakers, vibrating motor, LED lights, etc. The I/O devices 1322 may receive input from a user and also display an output of the computation performed by the processor 1302. In this document, the terms “computer program product” and “computer-readable medium” may be used generally to refer to media such as, for example, the memory 1306, the storage devices 1308, the removable storage unit 1314, or signal(s) on the channel 1320. These and other forms of computer-readable media may be involved in providing one or more sequences of one or more instructions to the processor 1302 for execution. Such instructions, generally referred to as “computer program code” (which may be grouped in the form of computer programs or other groupings), when executed, enable the computing system 1300 to perform features or functions of embodiments of the present invention.

In an embodiment where the elements are implemented using software, the software may be stored in a computer-readable medium and loaded into the computing system 1300 using, for example, the removable storage unit 1314, the media drive 1310 or the communications interface 1318. The control logic (in this example, software instructions or computer program code), when executed by the processor 1302, causes the processor 1302 to perform the functions of the invention as described herein.

Thus, the disclosed method and system try to overcome the technical problem of stitching anatomical images. The disclosed method and system may receive a first image and a second image. The first image corresponds to a first anatomical region and the second image corresponds to a second anatomical region. The first image may include an overlapping region with the second image. For each of a plurality of pixel rows of the first image selected from bottom to top, and for each of a plurality of pixel rows of the second image selected from top to bottom, the disclosed method and system may further cumulatively create a region of interest (ROI) from a first image pixel row with a second image pixel row until the ROI comprises each of the plurality of pixel rows of each of the first image and the second image. Further, the disclosed method and system may calculate an intensity difference between each of a plurality of first image ROI pixels and each of a corresponding plurality of second image ROI pixels. Further, the disclosed method and system may determine a mean intensity difference for the ROI based on the calculated intensity difference. Finally, the disclosed method and system may identify an overlapping ROI from a plurality of ROIs based on the mean intensity difference. The overlapping ROI may include a minimum mean intensity difference.

As will be appreciated by those skilled in the art, the techniques described in the various embodiments discussed above are not routine, or conventional, or well understood in the art. The techniques may provide for stitching of marker-free images. The techniques may further provide a comprehensive view of a larger anatomical region (for example, the spine) by seamlessly stitching the cervical image, the thoracic image, and the lumbar image. The techniques may provide a holistic view for accurately diagnosing conditions that may span multiple anatomical regions (for example, scoliosis, spinal tumors, degenerative disc diseases, or the like).

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The specification has described method and system for stitching anatomical images. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A method for stitching anatomical images, the method comprising:
receiving, by a computing device, a first image and a second image, wherein the first image corresponds to a first anatomical region and the second image corresponds to a second anatomical region, and wherein the first image comprises an overlapping region with the second image;
for each of a plurality of pixel rows of the first image selected from bottom to top, and for each of a plurality of pixel rows of the second image selected from top to bottom,
cumulatively creating, by the computing device, a region of interest (ROI) from a first image pixel row with a second image pixel row until the ROI comprises each of the plurality of pixel rows of each of the first image and the second image;
calculating, by the computing device, an intensity difference between each of a plurality of first image ROI pixels and each of a corresponding plurality of second image ROI pixels; and
determining, by the computing device, a mean intensity difference for the ROI based on the calculated intensity difference; and
identifying, by the computing device, an overlapping ROI from a plurality of ROIs based on the mean intensity difference, wherein the overlapping ROI comprises a minimum mean intensity difference.

2. The method of claim 1, comprising:
identifying a horizontal shift between the first image and the second image;
realigning the second image with the first image based on the identified horizontal shift; and
stitching the first image with the second image to obtain a stitched image.

3. The method of claim 2, wherein identifying the horizontal shift comprises:
aligning the first image with the second image to obtain an initial alignment, wherein the initial alignment comprises the overlapping ROI of the first image superimposed upon the overlapping ROI of the second image;
iteratively for each horizontal direction,
shifting the overlapping ROI of the second image by a pixel column towards the horizontal direction from the initial alignment to obtain a shifted alignment until the second image is completely unaligned with the first image, wherein the shifted alignment comprises one or more outlying pixel columns;
generating a difference image from the shifted alignment, wherein the one or more outlying pixel columns are excluded in the difference image; and
calculating a mean intensity of the difference image;
for each horizontal direction, determining a direction-wide minimum mean intensity from a plurality of mean intensities corresponding to a plurality of difference images obtained for the horizontal direction;
determining an overall minimum mean intensity from each of direction-wide minimum mean intensities; and
realigning the first image and the second image based on the overall minimum mean intensity.

4. The method of claim 2, wherein stitching the first image with the second image comprises:
obtaining a first part of the overlapping ROI from the first image and a second part of the overlapping ROI from each of the first image and the second image, wherein the first part and the second part are mutually exclusive;
blending the second part of the first image into the second part of the second image through the blending technique to obtain a blended second part; and
modifying a joining pixel row of the blended second part and the first part based on an average of adjacent pixel rows to the joining pixel row to obtain a stitched image.

5. The method of claim 1, comprising pre-processing the first image and the second image using one or more pre-processing techniques, wherein pre-processing the first image and the second image comprises, at least one of:
calculating an image mean intensity of each of the first image and the second image;
determining a maximum image mean intensity from the image mean intensity of each of the first image and the second image;
calculating a contrast factor of each of the first image and the second image based on the maximum image mean intensity;
adjusting a contrast of each of the first image and the second image based on the contrast factor; and
applying a histogram equalization technique to each of the first image and the second image.

6. A system for stitching anatomical images, the system comprising:
a processor; and
a memory communicatively coupled to the processor, wherein the memory stores processor instructions, which when executed by the processor, cause the processor to:
receive a first image and a second image, wherein the first image corresponds to a first anatomical region and the second image corresponds to a second anatomical region, and wherein the first image comprises an overlapping region with the second image;
for each of a plurality of pixel rows of the first image selected from bottom to top, and for each of a plurality of pixel rows of the second image selected from top to bottom,
cumulatively create a region of interest (ROI) from a first image pixel row with a second image pixel row until the ROI comprises each of the plurality of pixel rows of each of the first image and the second image;
calculate an intensity difference between each of a plurality of first image ROI pixels and each of a corresponding plurality of second image ROI pixels; and
determine a mean intensity difference for the ROI based on the calculated intensity difference; and
identify an overlapping ROI from a plurality of ROIs based on the mean intensity difference, wherein the overlapping ROI comprises a minimum mean intensity difference.

7. The system of claim 6, wherein the processor instructions, on execution, cause the processor to:
identify a horizontal shift between the first image and the second image;
realign the second image with the first image based on the identified horizontal shift; and
stitch the first image with the second image to obtain a stitched image.

8. The system of claim 7, wherein to identify the horizontal shift, the processor instructions, on execution, cause the processor to:
align the first image with the second image to obtain an initial alignment, wherein the initial alignment comprises the overlapping ROI of the first image superimposed upon the overlapping ROI of the second image;
iteratively for each horizontal direction,
shift the overlapping ROI of the second image by a pixel column towards the horizontal direction from the initial alignment to obtain a shifted alignment until the second image is completely unaligned with the first image, wherein the shifted alignment comprises one or more outlying pixel columns;
generate a difference image from the shifted alignment, wherein the one or more outlying pixel columns are excluded in the difference image; and
calculate a mean intensity of the difference image;
for each horizontal direction, determine a direction-wide minimum mean intensity from a plurality of mean intensities corresponding to a plurality of difference images obtained for the horizontal direction;
determine an overall minimum mean intensity from each of direction-wide minimum mean intensities; and
realign the first image and the second image based on the overall minimum mean intensity.

9. The system of claim 7, wherein to stitch the first image with the second image, the processor instructions, on execution, cause the processor to:
obtain a first part of the overlapping ROI from the first image and a second part of the overlapping ROI from each of the first image and the second image, wherein the first part and the second part are mutually exclusive;
blend the second part of the first image into the second part of the second image through the blending technique to obtain a blended second part; and
modify a joining pixel row of the blended second part and the first part based on an average of adjacent pixel rows to the joining pixel row to obtain the stitched image.

10. The system of claim 6, wherein the processor instructions, on execution, cause the processor to pre-process the first image and the second image using one or more pre-processing techniques, wherein to pre-process the first image and the second image, the processor is configured to, at least one of:
calculate an image mean intensity of each of the first image and the second image;
determine a maximum image mean intensity from the image mean intensity of each of the first image and the second image;
calculate a contrast factor of each of the first image and the second image based on the maximum image mean intensity;
adjust a contrast of each of the first image and the second image based on the contrast factor; and
apply a histogram equalization technique to each of the first image and the second image.
